(19) United States
(12) Plant Patent Application Publication
(10) Pub. No.: US 2020/0037483 P1
(43) Pub. Date: Jan. 30, 2020

(54) OLIVE TREE NAMED 'I-15'

(50) ;

(71) Applicant: Todolivo S.L., Córdoba (ES)

(72) Inventors: Luis Rallo Romero, Córdoba (ES); Diego Barranco Navero, Córdoba (ES); Concepción Muñoz DÍEZ, Córdoba (ES); Carlos Trapero RAMÍREZ, Córdoba (ES); Pedro Valverde CABALLERO, Córdoba (ES); José María GOMEZ PORRAS, Córdoba (ES)

(73) Assignee: Todolivo S.L., Córdoba (ES)

(21) Appl. No.: 16/602,027

(22) Filed: Jul. 24, 2019

(30) Foreign Application Priority Data
Jul. 25, 2018 (QZ) 2018/1938

(51) Int. Cl. A01H 6/00

(52) U.S. Cl. PLT/158

(57) ABSTRACT

A new and distinct variety of olive tree, herein referred to by its cultivar name, ‘I-15’, is provided which forms an upright to spreading tree growth habit. Fruit with a medium weight is produced. The fruit exhibit black coloration and the fruit nipple is absent or weak. The stone of the fruit of the new variety is of medium weight.

Botanical/commercial classification: Latin name—Olea europaea L. Common name—Olive tree.

Varietal denomination: ‘I-15’.

### CROSS-REFERENCE TO RELATED APPLICATIONS

This application claims priority to Plant Breeders' Right Application Number 2018/1938, which was filed at Community Plant Variety Office in the European Union on Jul. 25, 2018, the contents of which are hereby incorporated by reference for all purposes.

### SUMMARY OF THE INVENTION

The new variety of olive tree of the present invention was created by a controlled cross in Spain wherein two parents which previously had been studied were crossed in the hope that they would contribute the desired characteristics. The female parent (i.e., seed parent) of the new variety was the ‘ARBOSANA’ variety. The male parent (i.e., pollen parent) was the ‘KORONEIKI’ variety. The parentage can be summarized as follows:

‘ARBOSANA’ x ‘KORONEIKI’.

The seeds resulting from the above pollination were sown and small plants were obtained which were physically and biologically different from each other. Selective study resulted in the identification of a single plant of the new variety.

It was found that the new olive tree of the present invention possesses the following combination of characteristics:

(a) forms an upright to spreading growth habit, and

(b) produces fruit with medium weight, which are colored black at full maturity.

The new variety well meets the needs of the horticultural industry.

The new variety can be readily distinguished from related similar varieties.

The new variety has been found to undergo asexual propagation in Spain by vegetative cuttings. Asexual propagation by vegetative cutting in Spain has shown that the characteristics of the new variety are stable and are strictly transmissible by such asexual propagation from one generation to another. Accordingly, the new variety undergoes asexual propagation in a true-to-type manner.

The new variety has been named ‘I-15’.

### BRIEF DESCRIPTION OF THE PHOTOGRAPH

The accompanying photographs show as nearly true as it is reasonably possible to make the same, in a color illustration of this character, typical specimens of the plant parts of the new variety.

FIG. 1 illustrates specimens of fruit.

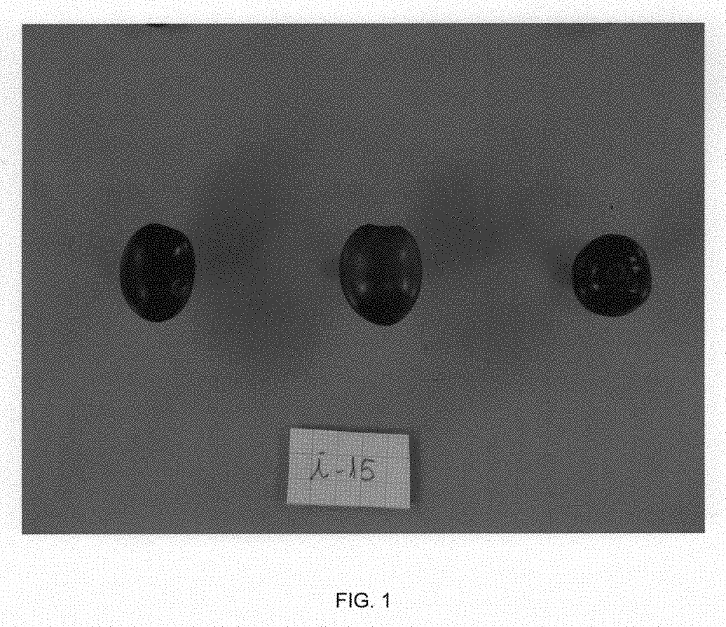

### DETAILED BOTANICAL DESCRIPTION

Plant:
   Habit.—Upright to spreading.
Fruit:
   Weight.—Medium.
   Color.—At full maturity is black.
   Symmetry.—Weakly asymmetric.
   Shape at Apex.—Rounded.
   Nipple.—Absent or weak.
   Stone.—Ratio length/width: moderately elongated. — weight medium. — mucron: present. — rugosity of surface: medium.

The new ‘I-15’ variety has not been observed under all possible environmental conditions to date. Accordingly, it is possible that the phenotypic expression may vary somewhat with changes in light intensity and duration, cultural practices, and other environmental conditions.

We claim:

1. A new and distinct variety of olive tree characterized by the following combination of characteristics:
(a) forms an upright to spreading growth habit, and
(b) produces fruit with medium weight, which are colored black at full maturity;
substantially as herein shown and described.
